(19) United States
(12) Patent Application Publication
(10) Pub. No.: US 2009/0252795 A1
(43) Pub. Date: Oct. 8, 2009

(54) Bioceramic scaffolds for tissue engineering

(76) Inventor: Stuart K.J. Smyth, Shelburne, VT (US)

Correspondence Address: STUART K. J. SMYTH, P.O. BOX 1059, Shelburne, VT, 05482, US

(21) Appl. No.: 12/157,121

(22) Filed: Jun. 9, 2008

Related U.S. Application Data
(60) Provisional application No. 61/123,089, filed on Apr. 7, 2008.

(51) Int. Cl. A61K 9/00; A61K 39/00; C12N 5/06

(52) U.S. Cl. 424/484; 424/184.1; 435/395; 435/402

(57) ABSTRACT

This invention relates to bioinert microporous bioceramic scaffolding, matrices or spheres comprising a particulate microporous bioinert ceramic material, which is a primary structure, and in the form of scaffolds, matrices or spheres, having interconnected pores. Pores on the surface of the spheres, matrices or scaffolds are connected to pores inside the spheres, matrices or scaffolds via blow-holes and the internal pores are in turn interconnected, so that in addition to a high porosity, the spheres, matrices or scaffolds will provide for a highly permeable, biosupportive environment for organic and/or inorganic substances, gases and/or liquids. The pores on the surface of the spheres, matrices or scaffolds are connected to pores inside the spheres, matrices or scaffolds via blow-holes and the internal interconnected pores within the highly porous microarchitecture or nanostructure(s) of the spheres, matrices or scaffolds will therefore prove conducive too, complimentary or suitable for, hosting such organic and/or inorganic substances, nanotechnologies, nanomaterials, gases or liquid that may be introduced, impregnated, activated, mixed, coated, transmitted or blended; and those various substances may be: resorbable bioceramic, polymers, copolymers, adhesives, hydrogels, collagen based materials, carbon based materials, metals, minerals, alternate corundum based materials, nanotechnologies, various forms of therapeutic energy, as well as various chemicals, chemoattractors and pharmaceutical agents or vaccine materials suitable for tissue treatment, tissue engineering, healing, regeneration/integration or repair of tissues within the body in order to ultimately restore tissue integrity, stability, viability, vitality, volume, mass, density, support, strength, purpose, design and function(s).

### CROSS REFERENCED TO RELATED APPLICATION

The present application is related to Provisional Application U.S. 61/123,089 and cross referenced to U.S. application Ser. No. 293707 filed Dec. 5, 2005 and Published Jun. 7, 2007 under United States Application 20070128244.

### INTRODUCTION

This invention relates to microporous bioceramic scaffolding, matrices or spheres comprising a particulate microporous bioinert ceramic material (a primary structure) and in the form of scaffolds, matrices or spheres, having interconnected pores. Pores on the surface of the spheres, matrices or scaffolds are connected to pores inside the spheres, matrices or scaffolds via blow-holes and the internal pores are in turn interconnected, so that in addition to a high porosity, the spheres, matrices or scaffolds will provide for a highly permeable, biosupportive environment for organic and/or inorganic substances, gases and/or liquids. This invention further relates to pores on the surface of the spheres, matrices or scaffolds that are connected to pores inside the spheres, matrices or scaffolds via blow-holes and the internal interconnected pores within the highly porous microarchitecture or nanostructure(s) of the spheres, matrices or scaffolds will therefore prove conducive too, complimentary or suitable for, hosting such organic and/or inorganic substances, nanotechnologies, nanomaterials, gases or liquid which may be introduced, impregnated, activated, mixed, coated, transmitted or blended. This invention further relates to those various substances which may be resorbable bioceramic, polymers, copolymers, adhesives, hydrogels, collagen based materials, carbon based materials, metals, minerals, alternate corundum based materials, nanotechnologies, various forms of therapeutic energy, as well as various chemicals, chemoattractors and pharmaceutical agents or vaccines suitable for tissue treatment, tissue engineering, healing, regeneration/integration or repair of tissues within the body.

### BACKGROUND TO THE INVENTION

There is an ongoing requirement for the treatment, healing, regeneration/integration or repair of tissues in the body, particularly where such materials are cell and tissue receptive. It is desirable for tissue regeneration/integration, replacement or repair for the scaffolding, matrices or spheres to provide a highly permeable microporous and interconnected microporous microarchitecture for revascularization, the proliferation of cells and tissue and thus the treatment, engineering, healing, regeneration/integration or repair of tissues and in order to ultimately restore tissue integrity, stability, volume, mass, density, support, strength, purpose, design and function. The choice of scaffolding, matrixes or spheres is crucial to enable the possibility for revascularization and proliferation of cells for the engineering, healing, regeneration/integration or repair of tissues. Current ceramic scaffolds, matrices or spheres are made by conventional ceramic scaffold, matrix or sphere fabrication methods, and most importantly they do not permit for the penetration of substances, gases or liquids which may aid or foster in the process of tissue engineering, regeneration/integration or repair of tissues. Current ceramic scaffolds, matrices or spheres lack the deep interconnected microporous environment conducive for internal revascularization and the proliferation of cells and tissues for engineering, healing, regeneration/integration or repair of tissues. In addition, current ceramic scaffolds, matrices or spheres may be too brittle or not exhibit the tensile strength capable of supporting porous and interconnected microporous microarchitecture or ceramic based host environment. Furthermore, these same current ceramic scaffolds, matrixes or spheres may lack the ability to allow for the internal introduction, impregnation, activation, coating, transmission or blending of various organic and/or inorganic substances which may be suitable for tissue engineering, healing, regeneration/integration or tissue repair. Substances such as resorbable bioceramic, polymers, copolymers, hydrogels, collagen based materials, carbon based materials, metals, minerals, alternate corundum based materials, nanotechnologies as well as various chemicals, chemoattractors, nanomaterials and/or pharmaceutical agents. It may be necessary for various substances to permeate deep around, into and/or throughout the microarchitecture of the scaffolds, matrices or spheres and therefore adhere, stick, assimilate, amalgamate, become like in porosity and interconnectivity, or take on a similar, partial or identical form, shape, design or structure to that of the porous microstructure ultimately which may not obstruct, rather support, a suitable biosupportive microporous interconnected microarchitecture for the treatment, engineering, healing, regeneration/integration or repair of various tissues. One of the principal methods used in tissue engineering involves growing the relevant cell(s) in vitro into the required three-dimensional (3D) organ or tissue. Cells lack the natural ability to grow in favored 3D orientations and thus define the anatomical shape of the tissue. Instead, they randomly migrate to form a (2D) layer of cells. However, 3D tissues are required and this is achieved by seeding the cells onto porous matrices, known as scaffolds, to which the cells attach and colonize. The scaffolds, matrices or spheres are therefore a very important component in tissue engineering, healing, regeneration/integration and tissue repair process.

Investigations into synthetic and natural inorganic ceramic materials, for example hydroxyapatite and tricalcium phosphate, as candidate scaffolding or matrices materials, have been aimed mostly at bone tissue engineering. This is primarily because these ceramics simulate the natural composition of bone and have osteoconductive properties. Other examples of materials utilized for scaffolding, matrices or spheres are the conventional synthetic polymers. However, the degradation of synthetic polymers, both in vitro and in vivo, releases acidic by-products which raises concerns that the scaffold microenvironment may not be ideal for tissue growth. Lactic acid is released from PLLA during degradation, reducing the PH, which further accelerates the degradation rate due to autocatalysis, resulting in a highly acidic environment adjacent to the polymer. Such an environment may adversely affect cellular function.

Cells attached to scaffolds are faced with several weeks of in vitro culturing before the tissue is suitable for implantation. Moreover, current synthetic polymers do not possess a surface chemistry which is familiar to cells that in vivo thrive on extracellular matrix made worthy of collagen, elastin, glycoproteins, proteoglycans, laminin and fibronectin. In contrast, collagen is the major protein constituent of the extracellular matrix and is recognized by cells as being chemostatic. Collagen scaffolds may present a more native surface relative to synthetic polymer scaffolds for tissue engineering purposes. However, like other natural polymers, collagen may elicit an immune response.

Most conventional ceramic scaffolding, matrixes or microsphere fabrication techniques are incapable of precisely controlling pore size, pore geometry, spatial distribution of pores and construction of internal channels within the scaffold. Typically, scaffolds produced by solvent-casting particulate leaching cannot guarantee interconnection of pores because this is dependent on whether the adjacent salt particles are in contact. Furthermore, skin layers that are formed during evaporation and agglomeration of salt particles make controlling the pore sizes difficult. For gas foaming, it has been reported that 10-30% of pores are interconnected. Non-woven fiber meshes have poor mechanical integrity.

Excluding gas foaming and salt molding, conventional scaffold fabrication techniques use organic solvents like chloroform and methylene chloride to dissolve synthetic polymers at some stage in the process. The presence of residual organic solvent is the most significant problem facing these techniques due to the risks of toxicity and carcinogenicity it poses to cells and tissues.

Conventional fabrication techniques produce scaffolds that are foam structures. Cells are then seeded and expected to grow into the scaffold. However, this approach has resulted in the in vitro growth of tissue with cross sections of less than 500 micrometers from the external surface. This may be due to the diffusion constraints in the foam. The pioneering cells cannot migrate deep into the scaffolding because the lack of nutrients and oxygen. The insufficient removal of the waste products that cell colonization at the scaffold periphery consumes also causes an effective barrier to the diffusion of oxygen and nutrients into the interior of the scaffold. Thus cells are only able to survive close to the surface. In this connection, it should be noted that no cell, except for chondrocytes, exists further than 25-100 micrometers away from a blood supply. The low oxygen requirement for cartilage may be the reason why only this tissue has been successfully grown in vitro to thick cross-sections i.e., greater than 1 mm using conventional scaffold fabrication techniques. Skin is a relatively 2D tissue and thus thick cross-sections of tissue are not required, thereby explaining the success of producing this tissue with conventional scaffold fabrication techniques. However, most other 3D tissues require a high oxygen and nutrient concentration and therefore an unobstructed microporous and biosupportive environment or microarchitecture.

Because of the limitations of current ceramic scaffolding materials, matrices or spheres, there exist the problem of providing a bioactive and/or biosupportive, bioinert ceramic scaffolding, matrices or spheres that will have some form of a porous and microporous internal highly permeable host environment to increase the mass transport of oxygen and nutrients deep within, and the removal of waste products from the scaffolding, matrices or spheres; and that may not have its microarchitecture obstructed or rendered biologically unsupportive as a host environment by various additional substances which may be required for the treatment, regeneration/integration and repair of cells and tissue. There is a demonstrated need for microporous ceramic scaffolding, matrices or spheres that will permit cells and tissue to migrate deep around, throughout, into, and from out of, the ceramic scaffolding, matrices or spheres and which will host various other supplementary therapies, nanotechnologies, organic and/or inorganic substances, nanomaterials, gases and/or liquids.

Current ceramic scaffolds, matrices or spheres lack the deep highly permeable microporous and interconnected microporous microarchitecture for internal revascularization which provides for the proliferation of cells and tissue for treatment, engineering, healing, regeneration/integration or repair of tissues. Furthermore, those same current ceramic scaffolds, matrices or spheres may lack the ability to allow for the impregnation, activation, coating or blending of various substances further required to aid in the various methods or processes involved in tissue treatment, tissue engineering, tissue healing, tissue regeneration/integration or tissue repair: with substances such as for example; resorbable bioceramic, polymers, copolymers, adhesives, hydrogels, collagen based materials, carbon based materials, nanotechnologies, nanomaterials, metals, minerals, alternate corundum based materials as well as various chemicals, chemoattractors and/or pharmaceutical agents. It may be necessary for various substances to permeate deep into and/or throughout the porous microarchitecture of the scaffolds, matrices or spheres and thus ultimately may adhere, stick, assimilate, amalgamate, become like, or conform partially or similar too, in terms of the characteristics specific to the porosity and interconnectivity of the primary ceramic scaffolds, matrices or spheres. Further these various substances may ultimately take on a similar, partial, somewhat similar or identical form, shape, design or structures which may not obstruct, rather support, the microporous interconnected microarchitecture or a biosupportive host environment for tissue treatment, engineering, healing, regeneration/integration or repair of tissues in order to ultimately restore tissue integrity, volume, mass, density, support, strength, purpose, design and function(s).

### OBJECTS OF THE INVENTION

It is therefore an object of the present invention to provide primary bioceramic scaffolding, matrices or spheres comprising a particulate microporous bioinert ceramic material, in the form of scaffolds, matrices or spheres, having interconnected pores. Pores on the surface of the spheres, matrices or scaffolds may be connected to pores inside the spheres, matrices or scaffolding via blow-holes and the internal pores are in turn interconnected, so that in addition to a high porosity, the spheres, matrices or scaffolds may have a high permeability to various organic and/or inorganic substances, gases and liquids which may aid in tissue treatments, tissue engineering, healing, regeneration/integration or tissue repair. It is therefore also within the same object of the present invention to create microporous scaffolds, matrixes or spheres that are permeable to organic and/or inorganic substances, nanotechnologies, gases and/or liquids and that provide a biosupportive host environment for the treatment, in-growth and/or revascularization and proliferation of cells and tissues in order to ultimately restore tissue integrity, volume, mass, density, support, viability, purpose, strength, design and function(s).

### SUMMARY OF THE INVENTION

According to the first aspect of the invention there may be a primary bioinert microporous ceramic scaffolding, matrices or spheres, having interconnected pores wherein the pores on the surface of the scaffolds, matrices or spheres may be connected to pores inside the scaffolding, matrices or spheres via blow-holes and the internal pores may in turn be interconnected, so that in addition to a high porosity, the scaffolding, matrices or spheres may have a high permeability to organic and/or inorganic substances, nanotechnologies, nanomaterials, gases and/or liquids.

Further, according to the invention, the microporous scaffolds, matrices or spheres porous and microporous microarchitecture may provide an unobstructed biosupportive host environment for the treatment and/or revascularization and the proliferation of cells and tissues.

A porous and interconnected microporous bioinert ceramic scaffolding, matrices or spheres wherein the porosity of the scaffolding, matrices or spheres may be 30% and 85% per volume depending upon the type of tissue treatment, engineering, healing, regeneration/integration or tissue repair.

A porous and interconnected microporous scaffolding, matrices or spheres wherein the diameters of the scaffolds, matrices or spheres may vary between 45 to 600 microns but may be smaller or larger depending upon the nature of, or desired outcome for tissue engineering, healing, regeneration/integration or tissue repair.

Yet further to the invention, a porous and microporous bioinert ceramic scaffolding, matrices or spheres wherein the pore diameters may be in the range of 0.3 to 15 micrometers, but may be smaller or larger depending upon the desired outcome or requirements for the treatment, tissue engineering, healing, regeneration/integration or tissue repair.

Yet further to the invention, the purpose, functionality and outcomes from the scaffolding, matrices or spheres may be a result of a codependent, interdependent, cooperative or combined biomechanical/biophysical relationship between the individual properties of the microarchitecture to the scaffolds, matrices or spheres in combination with the porous surface macroarchitecture of the scaffolds, matrixes or spheres; and as a result of the combined properties and characteristics of the entire or whole bioinert ceramic scaffolding, matrixes or spheres; the scaffolding, matrices or spheres may therefore provide an unobstructed biosupportive host environment for the treatment and/or revascularization and proliferation of cells and tissues in order to ultimately restore tissue integrity, volume, mass, density, support, strength, purpose, design and function(s).

Further, according to the invention, a microporous bioinert ceramic scaffolding, matrices or spheres having a porous and interconnected microporous form, shape, design or structure that may be suitable for the introduction, impregnation, activation, coating, transmission, mixing or blending of various organic and/or inorganic substances, nanotechnologies, nanomaterials, gases and/or liquids which may be suitable for the porous and microporous host environment, related processes and/or methods required for tissue engineering, healing, regeneration/integration or tissue repair in order to ultimately restore tissue integrity, volume, mass, density, support, purpose, design and function(s).

Further, according to the invention, porous and interconnected microporous bioinert ceramic scaffolding, matrices or spheres that may be suitable for the introduction, impregnation, activation, coating, transmission, mixing or blending of resorbable ceramics within and throughout the porous and interconnected microporous microarchitecture of the scaffolds, matrices or spheres and without obstructing, rather supporting, the biosupportive host environment for the treatment and/or revascularization and proliferation of cells and tissues.

Further, according to the invention, porous and interconnected microporous bioinert ceramic scaffolding, matrices or spheres that may be suitable for the introduction, impregnation, activation, coating, transmission, mixing or blending of polymers and/or copolymers within and throughout the porous and interconnected microporous microarchitecture of the scaffolds, matrices or spheres and without obstructing, rather supporting, the microporous host environment for the treatment and/or revascularization and proliferation of cells and tissues.

Further, according to the invention, porous and interconnected microporous bioinert ceramic scaffolding, matrices or spheres that may be suitable for the introduction, impregnation, activation, coating, transmission, mixing or blending of hydrogels, or other therapeutic gel based materials, within and throughout the porous and interconnected microporous microarchitecture of the scaffolds, matrices or spheres and without obstructing, rather supporting, the biosupportive host environment for the treatment and/or revascularization and proliferation of cells and tissues.

Further, according to the invention, porous and interconnected microporous bioinert ceramic scaffolding, matrices or spheres that may be suitable for the introduction, impregnation, activation, coating, transmission, mixing or blending of collagen based materials, or other organic tissue growth factors within and throughout the porous and interconnected microporous microarchitecture of the scaffolds, matrices or spheres and without obstructing, rather supporting, the biosupportive host environment for the treatment and/or revascularization and proliferation of cells and tissues.

Further, according to the invention, porous and interconnected microporous bioinert ceramic scaffolding, matrices or spheres that may be suitable for the introduction, impregnation, activation, coating, transmission, mixing or blending of carbon based materials such as diamond, or other conductive carbon based materials, within and throughout the porous and interconnected microporous microarchitecture of the scaffolds, matrices or spheres and without obstructing, rather supporting, the biosupportive host environment for the treatment and/or revascularization and proliferation of cells and tissues.

Further, according to the invention, porous and interconnected microporous bioinert ceramic scaffolding, matrices or spheres that may be suitable for the introduction, impregnation, activation, coating, transmission, mixing or blending of metals within and throughout the porous and interconnected microporous microarchitecture of the scaffolds, matrices or spheres without obstructing, rather supporting, the biosupportive host environment for the treatment and/or revascularization and proliferation of cells and tissues.

Further, according to the invention, porous and interconnected microporous bioinert ceramic scaffolding, matrices or spheres which may be suitable for the introduction, impregnation, activation, coating, transmission, mixing or blending of minerals and/or other corundum based materials, such as for example sapphire or ruby, within and throughout the porous and interconnected microporous microarchitecture of the scaffolds, matrices or spheres and without obstructing, rather supporting, the biosupportive host environment for the treatment and/or revascularization and proliferation of cells and tissues.

Further, according to the invention, porous and interconnected microporous bioinert ceramic scaffolding, matrices or spheres that may be suitable for the introduction, impregnation, activation, coating, transmission, mixing or blending of various chemicals, chemoattractors and/or other pharmaceutical agents within and throughout the porous and interconnected microporous microarchitecture of the scaffolds, matrices or spheres without obstructing, rather supporting, the biosupportive host environment conducive too or suitable for the treatment and/or revascularization and proliferation of cells and tissues.

Yet further to the invention, the porous and interconnected bioinert ceramic scaffolding, matrices or spheres may prove suitable for the application or utilization of targeted treatments or therapies that work in conjunction with or have an effect upon the porous and microporous microarchitecture and which may include organic and/or inorganic substances, nanotechnologies, nanomaterials, gases and/or liquids. Examples may be targeted drug therapies with the microporous ceramic, therapeutic energy applications with the microporous ceramic, targeted magnetic treatments with the microporous ceramic, targeted light or photodynamic therapy with the microporous ceramic, targeted FAR Infrared therapy with the ceramic, targeted thermodynamic or electromagnetic therapies with the microporous ceramic, targeted therapeutic surgical or minimally invasive treatments with the microporous ceramic or targeted nanotherapies, nanomaterials and nanotechnologies with the microporous ceramic as a means and/or methods to target and then treat, engineer, heal, regenerate/integrate or repair tissues.

Further, according to the invention, porous and interconnected microporous bioinert ceramic scaffolding, matrices or spheres which may be suitable for the introduction, impregnation, activation, coating, mixing, sending, retrieval, transmitting, receiving, reflecting or blending of light based therapy or energy, therapeutic thermodynamic energy, therapeutic electromagnetic energy, therapeutic radiotherapies, therapeutic nuclear based therapies, therapeutic ultrasonic based energy, acoustic treatment, FAR Infrared energy, magnetic energy, biocompatible semiconductor mechanisms, biocompatible suitable superconductor mechanisms within and throughout the porous and interconnected microporous microarchitecture of the scaffolds, matrixes or spheres and without obstructing, rather supporting, the biosupportive host environment for the treatment and/or revascularization and proliferation of cells and tissues.

Further, according to the invention, porous and interconnected microporous bioinert ceramic scaffolding, matrices or spheres which may be suitable for the introduction, impregnation, activation, coating, placement, integration, transmission, mixing or blending of therapeutic nanotechnologies, nanomaterials, nanotubes or other nanostructures within and throughout the porous and interconnected microporous microarchitecture of the scaffolds, matrices or spheres and without obstructing, rather supporting, the biosupportive host environment for the treatment and/or revascularization and proliferation of cells and tissues.

Yet further according to the invention, the porous and interconnected microporous microarchitecture of the scaffolding, matrices or spheres may have pores on the surface of the scaffolding, matrices or spheres may be connected to the pores inside the scaffolds, matrices or spheres via blow-holes, and the internal pores may be interconnected thus providing a possibly suitable porous and microporous microarchitecture for various organic or inorganic substances, nanotechnologies, nanomaterials, gases and/or liquids to permeate deep within and/or throughout the microarchitecture of the scaffolds, matrices or spheres. Thus, because of the bioinert ceramic microporous microarchitecture the organic and/or inorganic substances, nanotechnologies, nanomaterials, gases or liquids may adhere, stick, assimilate, amalgamate, become like in porosity and interconnectivity, or take on a similar, partial, or identical form, shape, design or structure thus not obstructing, rather supporting, the necessary conditions for a biosupportive microporous interconnected microarchitecture capable of hosting an environment suitable to the treatment and/or revascularization and proliferation of cells and tissues.

Further to the invention, the primary microporous ceramic scaffolds, matrices or spheres may be selected from a group of ceramic materials consisting of sintered aluminum oxide (alumina), zirconium oxide (zirconia), or combinations thereof, or hydroxyapatite.

Yet further to the invention, a precipitating resorbable material such as, and for example, tricalcium phosphate or calcium phosphate may be introduced, impregnated, coated, transmitted or mixed into and throughout the porous and interconnected microporous microarchitecture thereby bioactivating the internal microporous microarchitecture and host environment of the scaffolds, matrices or spheres which may be suitable for purposes relating to tissue treatment, tissue engineering, healing, regeneration/integration or tissue repair.

Further to the invention, wherein the primary bioinert ceramic scaffolding, matrixes or spheres may be resorbed by the body, shatter, break, split, splinter, burst, be dissolved, become altered, become different in anyway or changed from the scaffolds, matrices or spheres original state prior to their bioactivation and/or their introduction into the body.

Yet further to the invention, a primary bioinert ceramic scaffolds, matrixes or spheres which may be resorbed by the body, shatter, break, split, splinter, burst, be dissolved or become altered or become different in any way or changed from their original state prior to being introduced into the body. As a result of the various actions which may alter in anyway, change or make different the specific primary bioinert ceramic scaffolding, matrices or spheres; the organic and/or inorganic substances, nanotechnologies, nanomaterials, gases and/or liquids that may have been introduced, impregnated, activated, coated, transmitted, mixed or blended within and throughout the porous and microporous microarchitecture, and which had previously been enclosed, isolated or partially isolated by the entire macroarchitecture of the scaffolding, matrixes or spheres-may then become exposed, may develop into more direct or may make partial contact with a larger volume of tissue, larger presence or mass of tissue(s).

Further to the invention, as a result of a change, difference of, or alteration to the primary ceramic scaffolding, matrices or spheres, the in vivo exposure of a variety of internal organic and/or inorganic substances, nanotechnologies, nanomaterials, gases or liquids may retain an independent yet familiar, somewhat similar, partial or similar shape, form, design or structure to that of the original host ceramic porous and interconnected microporous scaffolding, matrices or spheres; and thus the same internal porous and microporous microarchitecture which may have been previously concealed, distanced from, or isolated by the scaffolds, matrices or spheres porous surface macroarchitecture, may eventually be made physically capable of coming into some form of a more direct, primary or altered contact with a larger volume of tissue(s) or larger presence of mass tissue(s) within the body because of such change, difference or alteration to the primary structure of the scaffolding, matrices or spheres.

Yet further to the invention, various organic and/or inorganic substances that may have been previously concealed by the macroarchitecture or porous surfaces of the scaffolding or spheres, may then become exposed directly or partially; and the various organic and/or inorganic substances (which may not be intrinsically have porous or microporous properties and characteristics, or may not intrinsically contain or be capable of independently supporting an interconnected microporous microarchitecture by nature of its individual characteristics and properties), may themselves finally become independently porous, independently assume a certain or specific degree of porosity, become somewhat independently porous, partially porous, become independently microporous or somewhat independently microporous in form, design, shape or structure wherein it may not have been previously possible to form, shape, design or structure such organic and/or inorganic substances into such independent and/or partially porous, independent and/or partially interconnected microporous scaffolds, matrices or spheres without the complete supporting structure or platform provided by the primary bioinert ceramic structure that was evident prior to any physical change or alteration of such primary ceramic scaffolds, matrices or spheres.

The primary bioinert microporous ceramic scaffolding, matrices or spheres may be prepared, but not limited to; a method to include the creation of a strong microporous ceramic for the purpose of tissue engineering, healing, regeneration/integration or tissue repair that may include the steps of:
1. Milling the pure precursor ceramic raw material into powder with particle size finer than 1 micrometer
2. Blending a combustible substance known in the trade of manufacturing porous structures into powder
3. Mixing the powder with water to form a paste or slurry
4) Forming solid spherical particles at a temperature of approximately 1100 C to 1600 C to form inert microporous ceramic spheres having pore sizes of between 0.3 to 15 micrometers (or may be more or less as required) and a diameter of between 45 to 600 micrometers (or may be more or (less), and, screening the spheres into pre-selected, preferably but not exclusively, narrow size fractions, for example 45 to 65 micrometer, 85 to 110 micrometer, 110 to 200 micrometer and so forth.

The applicants have found according to the present invention that the bioinert microporous ceramic material which comprise the primary ceramic scaffolding, matrices or spheres may meet, if not may exceed, most of the requirements for a ceramic scaffolding, matrices or spheres designed for the purposes of the treatment of tissue, tissue engineering, healing, regeneration/integration or tissue repair in order to ultimately restore tissue integrity, volume, mass, density, support, purpose, vitality, strength, design and function(s).

The descriptions of the embodiments of the present invention as given above are for the understanding of the present invention. It will be appreciated further that the invention may not be limited to the particular embodiments described herein, but may be capable of various modifications, rearrangements and substitutes as will be apparent to those skilled in the art without departing from the scope of the disclosure.

1) A primary bioinert microporous ceramic scaffolding, matrices or spheres, having interconnected pores wherein the pores on the surface of the scaffolds, matrices or spheres are connected to pores inside the scaffolding, matrices or spheres via blow-holes and the internal pores are in turn interconnected, so that in addition to a high porosity, the scaffolding, matrices or spheres will have a high permeability to organic and/or inorganic substances, nanotechnologies, nanomaterials, various therapeutic forms of energy, gases and/or liquids.

2) A primary bioinert microporous scaffolding, matrices or spheres which according to claim 1, are prepared either in vitro and/or in vivo and utilized in conjunction with therapeutic synthetic and/or non-synthetic, organic and/or inorganic biomaterials, therapeutic energy, liquids, gases and various pharmaceutical preparations or vaccines in order to produce a direct or indirect therapeutic effect upon tissues within the body.

3) A primary bioinert microporous scaffolding, matrices or spheres, which according to claims 1-2, the microporous scaffolds, matrices or spheres provide a highly permeable, biosupportive and thus virtually non-migratory host environment for the treatment, in-growth, out-growth and/or revascularization and the proliferation of cells and tissues.

4) A primary bioinert microporous scaffolding which according to claims 1-3 wherein the porosity of the scaffolding, matrices or spheres is 30% and 85% per volume depending upon the tissue treatment, engineering, healing, regeneration/integration or tissue repair with the primary scaffold, matrix or sphere regaining its original state/form or structure as the coated or impregnated organic/non-synthetic or inorganic/synthetic materials, gases and/or liquids are resorbed and/or redirected by the actual targeted or surrounding tissues; thus potentially providing for a greater volume of tissue to infiltrate around, within, from and throughout the microporous microarchitecture of the primary bioinert scaffolding, matrices or spheres.

5) A primary bioinert microporous scaffolding, matrices or spheres which according to claims 1-4 wherein the diameters of the scaffolds, matrixes or spheres will vary between 45 to 600 microns but may be smaller or larger depending upon the nature of, desired or intended outcome for tissue engineering, healing, regeneration/integration or tissue repair.

6) A primary bioinert microporous ceramic scaffolding, matrices or spheres according to claims 1-5 wherein the pore diameters will be in the range of 0.3 to 15 micrometers, but may be smaller or larger depending upon the additional substances, the desired outcome or requirements for the treatment, tissue engineering, healing, regeneration/integration or tissue repair.

7) A primary bioinert microporous scaffolding, matrices or spheres according to claims 1-6 whereby the purpose, functionality and outcomes from the scaffolding, matrices or spheres is a result of a codependent, interdependent, cooperative or combined biomechanical/biochemical relationship between the individual properties and characteristics of the microarchitecture of the scaffolds, matrices or spheres in combination with the porous surface macroarchitecture of the scaffolds, matrices or spheres; and as a result of the combined properties and characteristics of the entire or whole bioinert ceramic scaffolding, matrixes or spheres; the scaffolding, matrices or spheres will therefore provide a highly permeable, unobstructed biosupportive host environment for the treatment and/or revascularization and proliferation of cells and tissues in order to ultimately restore tissue integrity, stability, volume, mass, density, support, strength, viability, vitality, purpose, design, strength and function(s).

8) A primary bioinert microporous scaffolding, matrices or spheres which according to claims 1-7 whereby, according to a specific type of tissue and/or tissue application, the actual size of each the scaffolds, matrices or spheres can be varied in size, dimension, sequence, and degree of porosity and the variance will contribute to an increased compressive strength of the entire macrostructure and when combining more than just one singular primary bioinert microporous scaffold, matrix or sphere.

9) A primary bioinert ceramic microporous scaffolding, matrices or spheres according to claims 1-8 whereby the bioinert ceramic microporous scaffolding, matrices or spheres having an interconnected porous form, nanostructure, shape, design, characteristics, properties or structure is suitable for the in vitro and in vivo introduction, impregnation, activation, coating, transmission, mixing and/or blending of various organic/non-synthetic, inorganic/synthetic substances, nanotechnologies, nanomaterials, gases and/or liquids which may be suitable for the related therapeutic processes and/or methods required for tissue engineering, healing, regeneration/integration or tissue repair in order to ultimately restore tissue integrity, tissue volume, tissue mass, tissue density, tissue support, strength and the purpose of the tissue and the tissue function(s).

10) A primary bioinert microporous scaffolding, matrices or spheres according to claims 1-9 whereby the porous and interconnected microporous bioinert ceramic scaffolding, matrixes or spheres is suitable or complimentary for the introduction, impregnation, activation, coating, transmission, mixing or blending of resorbable ceramics (e.g. Tricalcium Phosphate, Calcium Phosphates, Hydroxyapatite) around, within and throughout the porous and interconnected microporous microarchitecture of the scaffolds, matrices or spheres thereby rendering the resorbable ceramic material porous and achieving virtually similar characteristics, forms or structures to that of the primary bioinert microporous ceramic scaffolds, matrices or spheres; and without obstructing, rather supporting, the biosupportive host environment for the treatment and/or revascularization and proliferation of cells and tissues.

11) A primary bioinert microporous scaffolding, matrices or spheres according to claims 1-9 whereby the porous and interconnected microporous bioinert ceramic scaffolding, matrices or spheres is suitable or complimentary for the introduction, impregnation, activation, coating, transmission, mixing or blending of polymers and/or copolymers or adhesives around, within and throughout the porous and interconnected microporous microarchitecture of the scaffolds, matrices or spheres and without obstructing, rather supporting, the microporous host environment for the treatment and/or revascularization and proliferation of cells and tissues.

12) A primary bioinert microporous scaffolding, matrices, or spheres according to claims 1-9 whereby the porous and interconnected microporous bioinert ceramic scaffolding, matrices or spheres is suitable or complimentary for the introduction, impregnation, activation, coating, transmission, mixing or blending of hydrogels, or other therapeutic gel based materials, around, within and throughout the porous and interconnected microporous microarchitecture of the scaffolds, matrices or spheres and without obstructing, rather supporting, the biosupportive host environment for the treatment and/or revascularization and proliferation of cells and tissues.

13) A primary bioinert microporous scaffolding, matrices or spheres according to claims 1-12 whereby the porous and interconnected microporous bioinert ceramic scaffolding, matrices or spheres is suitable or complimentary for the introduction, impregnation, activation, coating, transmission, mixing, reinforcing or blending of collagen based materials and/or other natural organic tissue regenerative preparations (specifically autologous myocytes, autologous fibroblasts and tissue allograths) around, within and throughout the porous and interconnected microporous microarchitecture of the scaffolds, matrices or spheres and without obstructing, rather supporting, the biosupportive host environment for the treatment and/or revascularization and proliferation of cells and tissues.

14) A primary bioinert scaffolding, matrices or spheres according to claims 1-13 whereby porous and interconnected microporous bioinert ceramic scaffolding, matrixes or spheres is suitable or complimentary for the introduction, impregnation, activation, coating, arranging, organizing, transmission, mixing or blending of carbon based materials such as diamond, or other conductive carbon based materials or nanostructures around, within and throughout the porous and interconnected microporous microarchitecture of the scaffolds, matrices or spheres and without obstructing, rather supporting, the biosupportive host environment for the treatment and/or revascularization and proliferation of cells and tissues.

15) A primary bioinert ceramic scaffolding, matrices or spheres according to claims 1-14 whereby the porous and interconnected microporous bioinert ceramic scaffolding, matrices or spheres that is suitable or complimentary for the introduction, impregnation, activation, coating, transmission, mixing, forming or blending of various metals around, within and throughout the porous and interconnected microporous microarchitecture of the scaffolds, matrices or spheres without obstructing, rather supporting, the biosupportive host environment for the treatment and/or revascularization and proliferation of cells and tissues.

16) A primary bioinert microporous ceramic scaffolding, matrices or spheres according to claims 1-15 whereby the porous and interconnected microporous bioinert ceramic scaffolding, matrices or spheres is suitable or complimentary for the introduction, impregnation, activation, coating, transmission, mixing or blending of minerals and/or other corundum based materials, such as sapphire or ruby, within and throughout the porous and interconnected microporous microarchitecture of the scaffolds, matrices or spheres and without obstructing, rather supporting, the biosupportive host environment for the treatment and/or revascularization and proliferation of cells and tissues.

17) A primary bioinert ceramic scaffolding, matrices or spheres according to claims 1-16 whereby the porous and interconnected microporous bioinert ceramic scaffolding, matrices or spheres is suitable or complimentary for the introduction, impregnation, activation, coating, transmission, mixing or blending of various chemicals, chemoattractors and/or other pharmaceutical agents or vaccines around, within and throughout the porous and interconnected microporous microarchitecture of the scaffolds, matrices or spheres.

18) A primary bioinert microporous ceramic scaffolding, matrices or spheres according to claims 1-17 whereby, the porous and interconnected bioinert ceramic scaffolding, matrices or spheres is suitable or complimentary for the application or utilization of targeted treatments or therapies that work in conjunction with, or have an effect upon, around, within and throughout the porous and microporous microarchitecture and which may include organic and/or inorganic substances, nanotechnologies, nanomaterials, gases and/or liquids. Examples are targeted drug therapies with the microporous ceramic, therapeutic energy applications with the microporous ceramic, targeted magnetic treatments with the microporous ceramic, targeted light or photodynamic therapy with the microporous ceramic, targeted FAR Infrared therapy with the ceramic, targeted thermodynamic or electromagnetic therapies or RF therapy with the microporous ceramic, targeted radiographic therapies, targeted therapeutic surgical or minimally invasive treatments with the microporous ceramic or targeted nanotherapies, nanomaterials and nanotechnologies with the microporous ceramic means and/or methods to diagnose, target and then treat, engineer, heal, regenerate/integrate or repair tissues.

19) A primary bioinert microporous ceramic scaffolding, matrices or spheres according to claims 1-18 whereby the porous and interconnected microporous bioinert ceramic scaffolding, matrixes or spheres provides for the introduction, impregnation, activation, coating, mixing, sending, retrieval, transmitting, receiving, reflecting, deflecting or blending of light base therapy or energy, therapeutic thermodynamic energy, therapeutic electromagnetic energy, therapeutic radiotherapies, therapeutic nuclear based therapies, therapeutic ultrasonic based energy, acoustic treatment, FAR Infrared, magnetic energy, biocompatible semiconductor mechanisms, biocompatible suitable superconductor mechanisms around, within and throughout the porous and interconnected microporous microarchitecture of the scaffolds, matrices or spheres and without obstructing, rather supporting, the biosupportive host environment for the treatment and/or revascularization and proliferation of cells and tissues.

20) A primary bioinert microporous ceramic scaffolding, matrices or spheres according to claims 1-19 whereby the primary microporous ceramic scaffolds, matrices or spheres will be selected from a group of ceramic materials consisting of sintered aluminum oxide (alumina), zirconium oxide (zirconia), or combinations thereof, or hydroxyapatite.

21) A primary bioinert microporous ceramic scaffolding, matrices or spheres according to claims 1-20 whereby a specified primary bioinert ceramic material comprising the scaffolding, matrixes or spheres can: be resorbed by the body, shatter, break, split, splinter, burst, be dissolved, become altered, become different in any way or changed from the scaffolds, matrices or spheres original state in vitro or prior to their bioactivation and/or their introduction into the body.

22) A primary bioinert microporous ceramic scaffolds, matrices or spheres according to claims 21 whereby the scaffolding, matrices or spheres can: be resorbed by the body, shatter, break, split, splinter, burst, be dissolved or become altered or become different in any way or changed from their original state (in vitro) or prior to being introduced into the body, or in vivo; and as a result of the various actions which can alter in anyway, change or make different the specific primary bioinert ceramic scaffolding, matrices or spheres; the organic and/or inorganic substances and/or any additional substances, nanotechnologies, nanomaterials, gases and/or liquids that have been introduced, impregnated, activated, coated, transmitted, mixed or blended around, within and throughout the porous and microporous microarchitecture, and which had previously been enclosed, isolated or partially isolated by the combined macroarchitecture and/or microarchitecture of the scaffolding, matrices or spheres will then become partially or fully exposed developing into codependent or altogether independent structures and making either direct or partial contact with a larger volume or mass of tissue(s).
